(19) United States
(12) Patent Application Publication
(10) Pub. No.: US 2024/0100290 A1
(43) Pub. Date: Mar. 28, 2024

(54) EXPIRATORY FLOW CONTROL

(71) Applicant: Covidien LP, Mansfield, MA (US)

(72) Inventors: Michal Marach, Erie, CO (US); Matthew J Phillips, Carlsbad, CA (US); Karen Ann Phillips, Paso Robles, CA (US)

(73) Assignee: Covidien LP, Mansfield, MA (US)

(21) Appl. No.: 18/471,646

(22) Filed: Sep. 21, 2023

Related U.S. Application Data
(60) Provisional application No. 63/409,519, filed on Sep. 23, 2022.

(51) Int. Cl. A61M 16/20

(52) U.S. Cl. CPC A61M 16/203; A61M 2016/0036

(57) ABSTRACT

Systems and methods for controlling an exhalation valve to increase flow resistance during exhalation. An example method includes delivering breathing gases for a first breath; controlling the expiratory valve according to a first closure profile during a first exhalation phase of the first breath; detecting, by the flow sensor, an exhaled gas flow rate during the first exhalation phase; determining that the exhaled gas flow rate during the first exhalation phase is indicative of a collapsed airway during the first exhalation phase; based on determining that the exhaled gas flow rate is indicative of the collapsed airway, setting a second closure profile of the expiratory valve for a second exhalation phase wherein the second closure profile of the expiratory valve increases airflow resistance for the second exhalation phase of a second breath; and controlling the expiratory valve according to the second closure profile during the second exhalation phase.

### CROSS-REFERENCE TO RELATED APPLICATIONS

This application claims the benefit of U.S. Provisional Application No. 63/409,519 filed Sep. 23, 2022, entitled “Expiratory Flow Control,” which is incorporated herein by reference in its entirety.

### BACKGROUND

Mechanical ventilation provides critical life support for patients requiring breathing assistance. A ventilator supplies oxygen or other breathing gases to a patient during inhalation and generally provides a low resistance airflow pathway for allowing the patient to exhale. For patients with certain types of breathing disorders, the low resistance airflow pathway of the ventilator may lead to obstruction or collapse of the patient's airway, which results in significantly restricted airflow and the inability to vent breathing gases from the patient's lungs.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In an aspect, the technology relates to a method for controlling an expiratory valve of a medical ventilator to improve ventilation. The method includes delivering, by the ventilator, breathing gases for multiple breaths; detecting a flow rate of the breathing gases during a first inhalation phase; based on the detected flow rate during the first inhalation phase, calculating an inhaled volume of breathing gases delivered during the first inhalation phase; detecting a flow rate of the breathing gases during a first exhalation phase; based on the detected flow rate during the first exhalation phase, calculating an exhaled volume of breathing gases delivered during the first exhalation phase; based on the exhaled volume being less than the inhaled volume by at least a threshold value, determining an indication of a collapsed airway; and based on determining the indication of the collapsed airway, adjusting a closure profile of the expiratory valve for a second exhalation phase wherein the closure profile of the expiratory valve increases airflow resistance for the second exhalation phase.

In an example, determining the indication of the collapsed airway is further based on a maximum-to-average exhaled flow rate difference. In another example, the second closure profile of the expiratory valve causes the expiratory valve to be at least 50% closed for at least a first 25% of the second exhalation phase. In still another example, the expiratory valve is external to the ventilator. In yet another example, the expiratory valve is positioned between a wye and a patient interface. In a further example, the closure profile of the expiratory valve causes the expiratory valve to be at least 25% closed for at least a first 50% of the second exhalation phase. In still another example, the method further includes detecting a flow rate of breathing gases for the second exhalation phase; based on the detected flow rate of breathing gases for the second exhalation phase, determining that the closure profile of the expiratory valve reduced an exhaled volume for the second exhalation phase; and based on the closure profile of the expiratory valve reducing the exhaled volume, reverting the closure profile of the expiratory valve to a prior closure profile.

In another aspect, the technology relates to a medical ventilation system including a ventilator including an inspiratory port and an expiratory port; an expiratory valve positioned external to the ventilator; a flow sensor; a processor; and memory storing instructions that when executed by the processor cause the medical ventilation system to perform operations. The operations include delivering breathing gases for a first breath; controlling the expiratory valve according to a first closure profile during a first exhalation phase of the first breath; detecting, by the flow sensor, an exhaled gas flow rate during the first exhalation phase; determining that the exhaled gas flow rate during the first exhalation phase is indicative of a collapsed airway during the first exhalation phase; based on determining that the exhaled gas flow rate is indicative of the collapsed airway, setting a second closure profile of the expiratory valve for a second exhalation phase wherein the second closure profile of the expiratory valve increases airflow resistance for the second exhalation phase of a second breath; and controlling the expiratory valve according to the second closure profile during the second exhalation phase.

In an example, the operations further include determining a maximum exhaled gas flow rate; determining an average exhaled gas flow rate; and wherein determining that the exhaled gas flow rate during the first exhalation phase is indicative of the collapsed airway is based on a difference between the maximum exhaled gas flow rate and the average exhaled gas flow rate. In still another example, the operations further include determining a delivered gas volume during an inhalation phase prior to the first exhalation phase; determining an exhaled gas volume during the first exhalation phase; and wherein determining that the exhaled gas flow rate during the first exhalation phase is indicative of the collapsed airway is based on a difference between the delivered gas volume and the exhaled gas volume. In yet another example, the second closure profile of the expiratory valve causes the expiratory valve to be at least 25% closed for at least a first 50% of the second exhalation phase. In a further example, the second closure profile of the expiratory valve causes the expiratory valve to be at least 50% closed for at least a first 25% of the second exhalation phase.

In another example, the operations further include detecting, by the flow sensor, an exhaled gas flow rate during the second exhalation phase; based on the exhaled gas flow rate during the second exhalation phase being higher than the exhaled gas flow rate for the first exhalation phase, setting a third closure profile of the expiratory valve for a third exhalation phase wherein the third closure profile of the expiratory valve increases the airflow resistance for the second exhalation phase compared to the first exhalation phase; and controlling the expiratory valve according to the third closure profile during the third exhalation phase. In yet another example, the operations further include detecting, by the flow sensor, an exhaled gas flow rate during the second exhalation phase; based on the exhaled gas flow rate during the second exhalation phase being lower than the exhaled gas flow rate for the first exhalation phase, setting a third closure profile of the expiratory valve for a third exhalation phase wherein the third closure profile of the expiratory valve decreases the airflow resistance for the second exhalation phase compared to the first exhalation phase; and controlling the expiratory valve according to the third closure profile during the third exhalation phase. In still another example, the operations further include detecting, by the flow sensor, an exhaled gas flow rate during the second exhalation phase; and based on the exhaled gas flow rate during the second exhalation phase being within a threshold range of the exhaled gas flow rate for the first exhalation phase, controlling the expiratory valve according to the second closure profile during a third exhalation phase.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a diagram illustrating an example medical ventilator system.

FIG. 2 is a set of graphs illustrating example breathing waveforms.

FIG. 3 is a set of graphs illustrating example breathing waveforms with an obstructed or collapsed airway condition.

FIG. 4 is a set of graphs illustrating example breathing waveforms with controlled improvement of an obstructed or collapsed airway condition.

FIG. 5 is a flow diagram illustrating a method for controlling a ventilator expiratory valve to improve an obstructed or collapsed airway condition.

FIG. 6 is a flow diagram illustrating a method for evaluating changes in physiologic breathing parameters following changes in airflow resistance.

### DETAILED DESCRIPTION

During the respiratory cycle, dynamic pressures within and around the lungs and associated airways drive breathing gases into and out of the lungs for gas exchange. These pressures are developed by a combination of the diaphragm, intercostal muscles, tissue elasticity, and other biological and anatomical mechanisms. The pressure developed within the lungs may be referred to as alveolar or intrapulmonary pressure, and the pressure exerted on the lungs and airways by surrounding tissues within the thorax may be referred to as intrapleural pressure. Intrapulmonary pressure falls below or rises above atmospheric pressure during inhalation and exhalation, respectively, resulting in airflow into or out of the lungs. As breathing gases travel through the airways, the gases encounter natural resistance, causing a pressure drop along the length of the airway. The point at which the pressure inside the airway equals the intrapleural pressure is referred to as the equal pressure point. Depending on where along the airway the equal pressure point is reached, further pressure drops along an airway within the lungs may result in airway obstruction or collapse because the intrapleural pressure would exceed the pressure within the airway. Obstruction or collapse of the airway may include a narrowing of the airway (minor airflow restriction), a partial collapse of the airway (moderate airflow restriction), or a full collapse of the airway (substantial airflow restriction).

A person with chronic obstructive pulmonary disease (COPD), or with other types of breathing disorders such as emphysema, can develop conditions within the lungs and airways that can lead to the obstruction or collapse of breathing passages during the exhalation phase of breathing. With COPD, conditions such as loss of elasticity in respiratory tissue or increased airway resistance can result in an increased pressure drop along the airway as the expelled air leaves the lungs. In a non-diseased airway, the natural resistance to airflow may result in a pressure drop that places the equal pressure point in a portion of the airway where cartilage reinforces the tissue, such as the trachea or bronchial branches, thereby avoiding airway collapse. However, in a person with COPD, the pressure drop along the obstructed airway may result in the equal pressure point moving more distally (i.e., towards or into deeper tissues within the lungs), where there is no cartilage support, resulting in further airway obstruction or the potential for airway collapse. An obstructed or collapsed airway prevents air or other breathing gases from being adequately exhaled from the lungs during the exhalation period, which affects overall respiratory efficacy. Over time the lungs may begin to overfill or fail to be completely exhausted, which may make breathing progressively more difficult, trapping excess carbon dioxide, reducing oxygenation, and hindering proper gas exchange.

A COPD patient experiencing airway obstruction or collapse may try to affect or improve this condition by altering how air is exhaled. For example, a COPD patient may perform “pursed-lip breathing,” where the mouth is held substantially closed (lips pursed) and exhalations are forced through a small opening in the lips. This maneuver increases the pressure within the airway, effectively moving the equal pressure point proximally (i.e., towards or into the upper airway), which helps hold the airways open during exhalation. The patient may then be able to more fully complete an exhalation. The action of pursing the lips increases the overall work of breathing since additional effort is required to force an exhaled breath through the narrow opening of the mouth. By increasing airflow resistance at the mouth, a COPD patient may gain some level of control over the obstructed airway, may improve total expiratory airflow, and may ultimately be able to breath more effectively.

As medical conditions dictate, it may be necessary for a COPD patient to receive breathing assistance from a mechanical ventilator where pursed-lip breathing may not be possible. During medical ventilation, a breathing circuit is established between a patient and a ventilator using a tubing system that includes an inspiratory limb coupled to an inspiratory port of the ventilator and an expiratory limb coupled to an expiratory port of the ventilator. An expiratory valve at the expiratory port may open and close to maintain or control pressures within the breathing circuit. During an exhalation phase of a breath, the expiratory valve normally opens to allow the patient to exhale with the low airflow resistance.

The low airflow resistance presented by a fully open expiratory valve reduces overall expiratory effort (e.g., work of breathing), which may be beneficial for patients not at risk of airway obstruction or collapse. However, this low airflow resistance may present a risk for ventilated COPD patients, who may be unable to engage in pursed-lip breathing as described above, and who benefit from some level of airflow resistance to help develop the pressure needed to keep the airways open and maintain airflow during exhalation. Consequently, COPD patients or patients with similar conditions may not receive the intended ventilation due to exhalation-induced obstruction or airway collapse.

The present technology may help increase expiratory breathing effectiveness in ventilated patients, such as patients with COPD or similar respiratory conditions, by reducing the potential for airway collapse. For example, the present technology controls exhalation airflow resistance via the expiratory valve of the ventilator to increase the airway resistance and work of breathing as compared to normal operations. In most patients, increasing the resistance and work of breathing would lead to discomfort and potentially less total volume exhaled by the patient. But, as discussed above, for some patients, the increased airflow resistance actually allows additional gas to be exhaled by the patient by maintaining the patency of the airways due to the increased pressure.

As additional detail, the present technology utilizes sensors within the ventilator system to measure the airflow, volume, pressure, and/or other physiologic breathing parameters. Based on these measurements, the ventilator may detect indications of airway obstruction or collapse. Upon detecting these indications during exhalation, the ventilator may adjust the closure profile (e.g., percentage and/or closure rate) of the expiratory valve to a partially opened state, which increases the airflow resistance and pressure in the airway as exhaled air passes through the valve, as compared to the valve being fully open. Due to the increased pressure generated by the expiratory valve, the airways of the patient may be held open, allowing the patient to complete an exhaled breath more fully and improve respiration. Using feedback from the sensors, the ventilator may further modify the closure profile of the expiratory valve by modifying the amount the expiratory valve is closed and the duration that the expiratory valve remains in that state, thereby modifying the airflow resistance as needed. Unlike positive end-expiratory pressure (PEEP), in which the ventilator may close the expiratory valve to maintain residual pressure in the airway at the end of exhalation, the present technology may permit the ventilator to maintain pressure earlier in the exhalation phase, and to maintain that pressure long enough for the patient to substantially complete the exhaled breath. Additional details are now provided by way of discussion of the drawings.

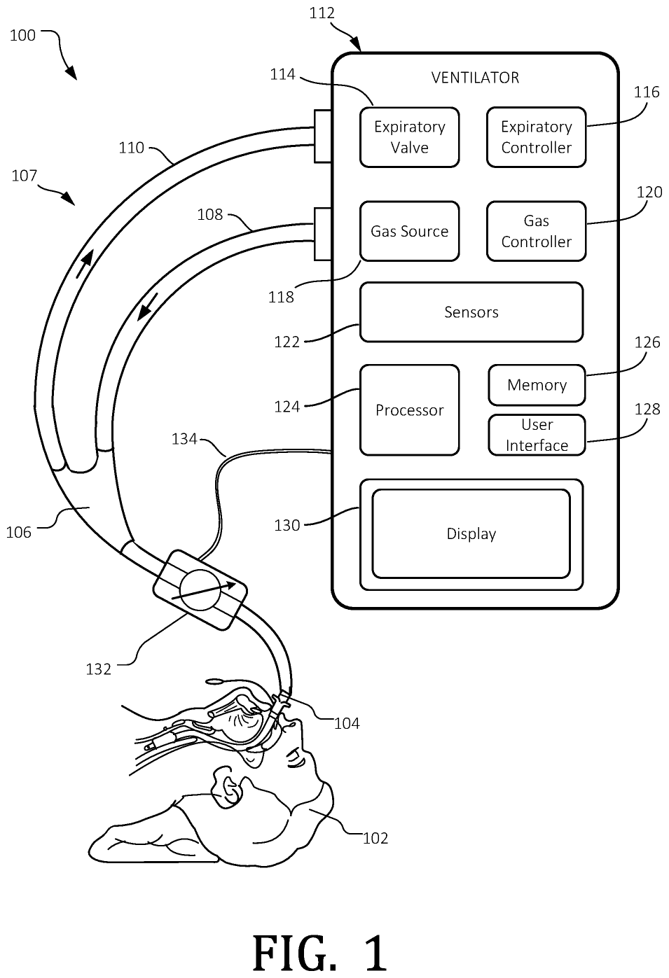

FIG. 1 is a diagram illustrating an example medical ventilation system 100, in which a patient 102 is connected to a ventilator 112 through a tubing system 107 and patient interface 104. The tubing system 107 (alternatively referred to as a patient circuit) may be a dual-limb circuit as shown in FIG. 1. The inhalation limb 108 is used to supply inhaled breathing gases from the ventilator 112 to the patient 102 during an inhalation, and the exhalation limb 110 is used to transport exhaled breathing gases from the patient 102 during an exhalation. As used in the context of exhalation, exhaled breathing gases may include carbon dioxide, unconsumed oxygen, or other gases exhaled from a patient 102 and vented by the ventilator 112. As used in the context of inhalation, inhaled breathing gases may include oxygen, anesthetic gases, or other gases supplied by the ventilator 112 and inhaled by a patient 102. The two limbs are joined at a patient wye 106, which is coupled to the patient interface 104. The patient interface 104 is depicted in FIG. 1 as an endotracheal tube but may another type of patient interface device such as a tracheostomy tube or other interface.

A gas source 118 and gas controller 120 may be coupled to the inhalation limb 108, within the ventilator 112. The gas source 118 and gas controller 120 may be a combination of gas compressors, blenders, accumulators, flow or pressure regulators, humidifiers, filters, valves, pneumatic actuators, gas storage apparatus, or other mechanisms used by the ventilator 112 to deliver inhaled breathing gases to a patient. The gas source 118 may include or be connected to an external gas supply, such as from a centralized oxygen or air supply system provided within a clinical setting, or from pressurized gas cylinders or other sources. The gas controller 120 may include the analog or digital circuitry needed for controlling motors, valves, actuators, or any of the other electrical and/or mechanical mechanisms listed above within the ventilator 112. The gas controller 120 may be communicatively coupled to the processor 124, or other systems or subsystems within the ventilator 112. In examples, the gas source 118 or gas controller 120 may be coupled to portions of the ventilator 112 associated with exhalation functions, such as expiratory valve 114 or expiratory controller 116.

The exhalation limb 110 may be coupled to the expiratory valve 114 or expiratory controller 116, within the ventilator 112. The expiratory valve 114 may be a mechanically, electrically, electromagnetically, or pneumatically operated diaphragm, balloon valve, scissor valve, ball valve, or any other type of valve mechanism capable of operating in any state of closure, ranging from fully open (minimal resistance to airflow) to fully closed (no airflow), or any state therebetween (resisted airflow). For instance, the expiratory valve may be a proportional solenoid (PSOL) valve. Depending on the type of valve, while in a fully open state, the expiratory valve 114 may exhibit airflow resistance equal to or less than the airflow resistance inherent to the tubing system 107, or other parts of the patient circuit. In examples, while in the fully open state, the expiratory valve 114 may add minimal or negligible airflow resistance to the total airflow resistance of the overall patient circuit. The expiratory valve 114 may be capable of achieving finely tunable levels of airflow resistance based on the degree to which the valve is closed/open. In examples, the expiratory valve 114 may open or close in discrete steps or may smoothly vary between different degrees of closure. For example, the level of closure of the expiratory valve 114 may be controlled to increase airflow resistance and limit airflow during exhalation to a degree that mimics pursed-lip breathing in non-ventilated COPD patients. The increased airflow resistance may increase pressure in the airway of the patient, which may help maintain airway patency and allow the patient to perform more fully completed exhalations.

In some examples, the expiratory valve 114 may be directly coupled to the exhalation limb 110, where exhaled breathing gases may flow directly from the exhalation limb 110 to the expiratory valve 114. In other examples, the airflow input of the expiratory valve 114 may be coupled to the exhalation limb 110 through other elements or mechanisms of the ventilator 112, such as additional valves, sensors or sensor modules, tube couplings or splitters, or other elements of the ventilator 112. In such examples, exhaled breathing gases may flow from the exhalation limb 110 into these elements of the ventilator 112, then to the expiratory valve 114. When partially or fully open, exhaled breathing gases may pass through the expiratory valve 114 and be expelled to atmosphere or other low-pressure terminus. In examples, the ventilator 112 may provide filtering elements (not depicted in FIG. 1) for removing pathogens, viruses, or other microorganisms that may be contained in the exhaled breathing gases before venting these gases to the atmosphere or other low-pressure terminus.

The expiratory valve 114 or expiratory port may include sensors disposed therein for measuring flow, pressure, temperature, or other physiologic breathing parameter associated with exhaled breathing gases. In examples, the expiratory valve 114 may be electrically coupled to other systems, subsystems, or elements within ventilator 112. For example, the expiratory valve 114 may provide connections for control signals, data signals, clock sources, or other signals, or may provide connections for electrical power supplied to the expiratory valve 114. In some examples, the expiratory valve may be coupled to other systems or elements within the ventilator 112 by wireless means. The expiratory valve 114 may include analog or digital circuitry, electrical or mechanical components, or any other elements or mechanisms needed for valve actuation, or to interface with other systems, mechanisms, or elements within or external to ventilator 112.

In some examples, a modularized external expiratory valve may be coupled to a portion of the exhalation limb 110, or some other portion of the tubing system 107, as a separate device from the ventilator 112. Such an external expiratory valve 132 may be used as an add-on component for those patients that the conditions discussed herein that are susceptible to airway collapse during exhalation. For example, the external expiratory valve 132 may be positioned between the wye 106 and the patient interface 104. The external expiratory valve 132 may include two ports to receive tubing and connect to the patient circuit 107. In other examples, the external expiratory valve may operate as a pinch or scissor valve to compress the tubing of the patient circuit to limit airflow or increase airflow resistance.

The external expiratory valve 132 may have the same or similar features and components for achieving varying degrees of valve closure as the expiratory valve 114. The external expiratory valve 132 may couple to the ventilator 112, which may control or interface with the external expiratory valve 132 via interface cable 134, as described herein. In other examples, the external expiratory valve may couple to another device or system that may be associated with the ventilator 112 or that may operate independently. For instance, the external expiratory valve 132 may include a controller, with a processor and memory, or be connected to a controller that allows the external expiratory valve 132 to perform the operations described herein.

The ventilator 112 may include an expiratory controller 116 that may interface with the expiratory valve 114 and/or other systems within, or external, to the ventilator 112. The expiratory controller 116 may provide control of the expiratory valve 114 via electrical, mechanical, pneumatic, or other means. The expiratory controller 116 may comprise a combination of analog or digital circuitry (e.g., passive components, digital logic, microcontrollers, or other type of circuit element), electromechanical components (e.g., relays or motors), mechanical mechanisms, pneumatic mechanisms (e.g., compressors, reservoirs, pumps, cylinders, pistons, or other types of pneumatic components), or any other type of component or mechanism used to control or interface with the expiratory valve 114. In examples, the expiratory controller 116 may incorporate feedback from sensors associated with the expiratory valve 114 or other sensors within the ventilator 112, such as sensors 122. In examples, the expiratory controller 116 may interface with other systems within the ventilator 112, such as a processor 124, memory 126, portions of the ventilator 112 associated with the inhalation limb 108, or any other system that may facilitate control of functions or features associated with exhalation. For instance, the expiratory controller 116 may adjust closure of the expiratory valve 114 based on control signals, instructions, or other information received from the processor 124. In other examples, the expiratory controller 116 may directly adjust closure of the expiratory valve 114 independently, without receiving control signals, instructions, or other information from other systems or elements within the ventilator 112.

The ventilator 112 may include different types of sensors 122 associated with operation of the ventilator 112. For example, sensors 122 may comprise a plurality of temperature, humidity, flow, pressure, gas concentration, pulse oximetry, or any other type of sensor associated with the measurement of the gas properties of the inhaled or exhaled breathing gases. In examples, sensors 122 may include a combination of electrical, mechanical, electromagnetic, thermal, optical or other sensor technologies that are capable of providing measurement data used by features, functions, or for the operation of ventilator 112. The sensors 122 may be distributed across a variety of locations within the ventilator 112, or the sensors 122 may otherwise not be collocated within the ventilator 112, as dictated by the function of each sensor. For example, one or more of the sensors 122 may be coupled to, or associated with breathing gases exchanged via the inhalation limb 108 or exhalation limb 110. In examples, one or more of the sensors 122 may be coupled to, or associated with electrical, mechanical, pneumatic, or other system or subsystem of the ventilator 112, including features or functions that are not directly associated with the measurement of breathing gases.

The sensors 122 may include one or more flow sensors. In examples, the flow sensors may be mass flow sensors, which measure the transfer of thermal energy to determine flow. For instance, the one or more flow sensors may be mass flow sensors packaged in integrated circuits (ICs) or may be a “hot wire” type of design. In other examples, the one or more flow sensors may be mechanical based, where the gas flow is determined by measurement of moving parts. For instance, flow may be determined by rotation of a sensor element, such as a paddle wheel or propeller, or flow may be determined by movement of some other element or mechanism. The one or more flow sensors may include sensing mechanisms such as infrared (IR), ultrasound, electromagnetic, capacitive, inductive, or other type of sensing mechanism. In examples, the one or more flow sensors may be any combination of the various technologies described.

The sensors 122 may include one or more pressure sensors. The pressure sensors may be any of a variety of possible pressure sensing solutions. For instance, the pressure sensor(s) may be based on a strain gauge, or a resistive, capacitive, inductive, micro electro-mechanical system (MEMS), or other type of sensing element. The pressure sensor(s) may be based on a deflection-type mechanism, such as a diaphragm or other deflectable element, or the one or more pressure sensors may be based on another type of pressure sensitive mechanism.

In some examples, one or more of the sensors 122 may include individual sensing transducers (e.g., a thermistor or other type of basic transducing element), while in other examples, one or more of the sensors 122 may include sensing systems, where additional signal conditioning or processing may be performed within the sensing system. For example, a sensing system of sensors 122 may include a sensor transducer, analog filters or other signal conditioning elements, an analog-to-digital converter (ADC), digital signal processing elements, or other elements grouped together in a common package or printed circuit board (PCB). The sensor 122 may include any combination of analog or digital circuitry (e.g., passive components, digital logic, microcontrollers, or other type of circuit element), or any other element required for measurement signal transduction and processing. In examples, the sensors 122 may further include elements or mechanisms required for the transmission of measurement data to other systems within ventilator 112.

In some examples, the sensors 122 may include sensors that are external to the ventilator 112. For example, flow or pressure sensors may be coupled to the tubing system 107 and may interface with the ventilator 112 to provide measurement data. The external sensors may interface with the ventilator 112 and/or components thereof.

Processing circuitry, such as processor 124, may include a combination of digital or analog circuitry for processing signals received from, or transmitted to, other systems, elements, or mechanisms within the ventilator 112, such as sensors 122, expiratory valve 114, or expiratory controller 116. The processor 124 may include one or more general purpose processors, microprocessors, microcontrollers, digital signal processors (DSPs), or other programmable circuits. In examples, the processor 124 may include a combination of commercially available components, or custom or semi-custom integrated circuits, such as application specific integrated circuits (ASICs). The processor 124 may include elements needed for control of, or communication with memory 126, user interface 128, or display 130, in addition to elements for controlling or interfacing with the breathing features or functions of the ventilator 112. The processor 124 may perform control, interface, communication, or other processing functions by executing instructions that are stored in the memory 126. The memory 126 may include RAM, ROM, electrically erasable programmable read-only memory (EEPROM), flash memory or other memory technology, CD-ROM, digital versatile disks (DVD) or other optical storage, magnetic cassettes, magnetic tape, magnetic disk storage or other magnetic storage devices, or other types of storage media, including externally connected storage media. The memory 126 may store instructions, that when executed by the processor 124, cause the system 100 to perform the operations described herein.

The processor 124 may be configured to execute code, run algorithms, or perform other types of analysis or operations on measurement data received from sensors 122. For example, the processor 124 may receive exhalation airflow data from a flow sensor associated with the exhalation limb 110 or expiratory valve 114, and the processor 124 may further receive sample timing information associated with the exhalation airflow data (such as from an ADC, clock source, or other source of timing information). The airflow data and sample timing data may be combined by the processor 124 to produce an estimate of the volume of gas inhaled or exhaled during each phase of the breathing cycle. The processor 124 may further monitor measurement data for trends in exhalation airflow, volume, or pressure over a number of breathing cycles, and detect conditions that may indicate airway obstruction, or full or partial airway collapse. The processor 124 may respond to these types of conditions by modulating the closure of the expiratory valve 114 to reduce airflow, increase airway pressure, and maintain airway patency. In examples, the processor 124 may provide data or control signals to the expiratory valve 114 or expiratory controller 116 that may cause actuation of the expiratory valve 114 in response to measurement data received from sensors 122.

The processor 124 may receive user input from a user interface 128. In examples, the user interface 128 may include buttons, knobs, dials, switches, or other forms of user-selectable input that may be made available to the user on the exterior of the ventilator 112. The user interface 128 may allow clinicians to configure the ventilatory features or functions of the ventilator 112 (e.g., tidal volume, respiratory rate, PEEP), determine the information to be displayed on the display 130, set alert conditions, store or retrieve operational settings to or from memory 126, store or retrieve measurement data to or from memory 126, or provide other types of input to the ventilator 112. In examples, the user interface 128 may be used to configure airflow resistance settings that are implemented by the expiratory valve 114 during exhalation. For example, the user interface 128 may allow a clinician to specify the degree of closure of the expiratory valve 114, the time duration(s) of the expiratory valve 114 closure, the level of airflow resistance developed by the expiratory valve 114, and/or other settings related to maintaining airway patency via the expiratory valve 114 during exhalation. In other examples, the user interface 128 allows a clinician to configure the ventilator 112 to automatically adjust expiratory valve airflow resistance in response to measurement data from sensors 122, such as through the processor 124 or expiratory controller 116.

In some examples, the user interface 128 may provide audible alerts, such as through a speaker (not depicted in FIG. 1) coupled to the user interface 128. For example, if a clinician has configured the ventilator 112 to sound an alert for low expiratory volume, and this condition is detected by elements within the ventilator 112 (such as by sensors 122 or processor 124), an audible alert may be sounded.

The processor 124 directs information to be displayed on the display 130. The information may include data collected or derived from the sensors housed within the ventilator 112 or information related to the respiratory status of the patient. In some examples, the display information may be in numeric or graphical form (e.g., airflow, volume, or pressure waveforms versus time). In other examples, the displayed information may include status or other operating information related to the ventilator 112. In examples where the display 130 is a touch-sensitive screen (e.g., a capacitive touch-sensitive screen), the processor 124 may receive user inputs from the display 130 and respond accordingly. The display 130 may be any of a light-emitting diode (LED) screen, a liquid-crystal display (LCD) screen, an organic LED (OLED) screen, or other display technology. In still other examples, the display 130 may include any type of visual indicator such as lamps, bulbs or strip light sources, or other types of discrete visual indicators. A combination of these indicators or display technologies, of display 130, may be used to provide a visual indication of an alert condition detected by the ventilator 112, or to display information to a clinician.

In operation, the ventilator 112 may detect indications of airway obstruction or collapse in a ventilated patient 102. In examples, the ventilator 112 detects insufficient gas flow as measured by sensors 122 and observed by the processor 124 or other elements of the ventilator 112. The ventilator 112 may further determine, through measurement data processing, that the volume of breathing gases delivered during the inhalation phase of a breath does not match the volume of gas being exhaled during the exhalation phase of the breath. To detect indications of airway obstruction or collapse, the ventilator 112 may observe sensor measurement data during inhalation, exhalation, or combinations of both breath phases. In some examples, the ventilator 112 utilizes sensor measurement data from external sensors to determine expiratory valve airflow resistance. In other examples, the ventilator 112 utilizes sensor measurement data from a combination of external and internal sensors (such as sensors 122) to determine expiratory valve airflow resistance. Indications of the insufficient airflow or asymmetric inhalation/exhalation volumes may be presented to a clinician via the display 130 and/or through an audible alert.

The ventilator 112 also addresses the exhalatory airflow and volume deficiencies by controlling the exhalation valve 114 to increase resistance during the exhalation phase of the breath as compared to a prior exhalation phase. For example, a clinician, may configure the ventilator 112, through user interface 128, to increase airflow resistance during exhalation via the expiratory valve 114, which may sustain pressure in the airway of the patient 102 (similar to purse-lipped breathing). In examples, the processor 124 or other element may then use measurement data collected from sensors 122 to detect the onset of exhalation in each breathing cycle and adjust the degree of closure of the expiratory valve 114. The clinician may further adjust expiratory valve airflow resistance based on measured changes in airflow and exhaled volume, as presented via the display 130.

In other examples, the ventilator 112 automatically detects trends in exhalation airflow, volume, pressure, or other physiologic breathing parameter, and automatically determines an appropriate level of airflow resistance needed to maintain airway patency. For example, processor 124 processes the data measured from sensors 122 and/or stored in memory 126 over one or more breathing cycles. The processor 124 may execute an algorithm for determining an initial airflow resistance setpoint for the expiratory valve 114 and may collect additional sensor data to analyze changes in exhalation that result from the increased airflow resistance. The processor 124 then adjusts airflow resistance (via expiratory valve 114) to further improve airflow or ventilation for the patient 102. In other examples, the processor 124 or other elements of ventilator 112 may determine the level of expiratory valve airflow resistance needed to mitigate or improve airway obstruction or collapse based on a combination of current sensor measurement data, stored sensor measurement data, pre-defined or pre-determined settings, stored population data, user profiles, statistical data or measures, or other type of information.

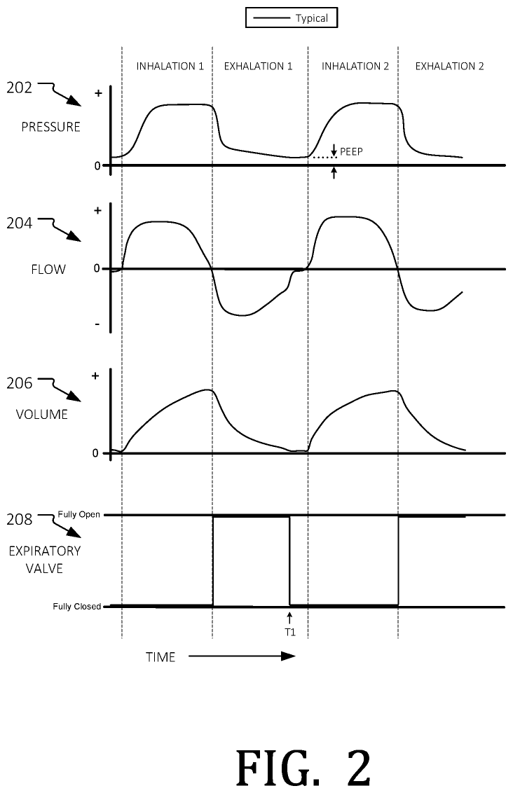

FIGS. 2, 3, and 4 present a progression of example breathing waveforms that illustrate how the present technology may improve respiration in mechanically ventilated patients with COPD or similar respiratory conditions. It should be noted that breathing waveforms, patterns, trends, and other features of breathing can vary considerably between patients and can vary over the course of many breathing cycles within the same patient. The presented waveforms are examples of many possible scenarios that may be presented by a ventilated patient. Waveform features illustrated in these figures may not be to scale, and portions may be magnified or emphasized for clarity of visualization to aid in the disclosure of the present technology. All waveforms presented herein depict physiologic breathing parameters (also referred to as gas properties) on a vertical axis and the progression of time on a horizontal axis. Waveforms representing the state of the respiratory valve over time are also presented. The axes of all waveform plots are presented without numeric units of measure since numeric units are not necessary to convey aspects of the present technology. Rather, the vertical axes are presented with +/−axis labels, to indicate positive or negative values in each physiologic breathing parameter, and the expiratory valve waveform is presented with axis labels indicating the closure state of the expiratory valve.

FIG. 2 illustrates a set of waveforms that represent two breathing cycles from a ventilated patient that may be considered examples of “normal” or “typical” breaths from a patient without a condition that would cause the airway collapses during exhalation discussed herein. Pressure waveform 202 depicts breathing gas pressure at a location within the breathing circuit or representative of pressure in the lungs. The pressure signal may represent pressure levels relative to atmospheric pressure, where “0” represents a pressure level equal to atmospheric pressure. The pressure waveform may be measured by the ventilator with one or more sensors (such as sensors 122). The pressure sensor(s) may be associated with the expiratory valve (such as expiratory valve 114 or external expiratory valve 132), exhalation limb (such as exhalation limb 110), inhalation limb (such as inhalation limb 108), gas source (such as gas source 118), gas controller (such as gas controller 120), or may be associated with another portion of the ventilator or patient breathing circuit. In examples, the ventilator uses data from a single pressure sensor or combines the measured data collected from a plurality of pressure sensors to form the pressure waveform 202.

Flow waveform 204 depicts gas flow into and out of the patient's lungs. Positive gas flow represents the flow of inhaled breathing gases into the lungs (such as the flow of oxygen or other supplied gases), while negative gas flow represents the flow of exhaled breathing gases out of the lungs (such as the flow of carbon dioxide and unconsumed oxygen). The flow waveform 204 may be measured by the ventilator with one or more sensors (such as sensors 122). In examples, the ventilator uses data from a single flow sensor or combines the measured data collected from a plurality of flow sensors to form the flow waveform 204.

Volume waveform 206 depicts the volume of breathing gases within a patient's lungs. The volume may be derived or calculated by the elements in the ventilator (such as processor 124) using flow sensor measurements and timing data to approximate the integral of the flow waveform 204. For instance, the rate of gas flow over time may be used to calculate the volume of gas that has been delivered (e.g., inhaled) and the volume of gas that has been exhaled.

Valve-state waveform 208 depicts the state of closure of the expiratory valve over time. The valve-state waveform 208 includes a high position that indicates the expiratory valve being fully open and a low position that indicates the expiratory valve is fully closed. While the valve-state waveform 208 shows the expiratory valve in only fully open and fully closed states, the expiratory valve may be positioned to be partially closed (e.g., between the fully open and fully closed states). In current practice, the ventilator may fully open the expiratory valve during exhalation, so that airflow resistance is minimized during this phase of each breathing cycle. As described, this low airflow resistance may reduce respiratory effort for many ventilated patients, but that low resistance may pose a risk of further airway obstruction or collapse for patients with COPD or similar conditions. During inhalation, the expiratory valve may fully close to prevent supplied oxygen or other breathing gases intended for the patient from being diverted through the exhalation limb.

During the first inhalation phase (Inhalation 1), the expiratory valve is fully closed, as shown in valve-state waveform 208. Breathing gases are supplied to the patient through the inhalation limb of the ventilator system, which can be seen by the positive gas flow in gas flow waveform 204. Airway pressure increases and reaches a maximum as air flows into the lungs, as shown in pressure waveform 202. As indicated by the volume waveform 206, as air flows into the lungs they begin to fill, reaching a maximum volume near the end of the first inhalation phase. Gas flow into the lungs begins to taper and reach zero near the end of the first inhalation phase, where the first exhalation phase (Exhalation 1) begins. Depending on the configuration of the ventilator, the volume and flow characteristics of delivered breathing gases may be preconfigured by a clinician and regulated by the ventilator along with the duration and general timing of the inhalation and exhalation phases.

As the ventilator transitions from the from the first inhalation phase (Inhalation 1) to the first exhalation phase (Exhalation 1), the expiratory valve transitions from fully closed to fully open to provide minimal airflow resistance during exhalation. For instance, for a typical patient, the least amount of airflow resistance allows for the patient to exhale with greater ease. The change in expiratory valve airflow resistance results in a precipitous drop in airway pressure as the airflow reverses, and breathing gases are exhaled from the lungs (negative flow in flow waveform 204) through the exhalation limb of the ventilator system. The volume of breathing gases in the lungs decreases in accordance with the gas flow, as depicted in volume waveform 206.

At time T1, depicted in valve-state waveform 208, the expiratory valve transitions from fully open to fully closed just prior to the onset of the next inhalation phase (Inhalation 2). With the expiratory valve closed, airflow out of the lungs through the exhalation limb of the ventilator system rapidly approaches zero, and a small residual volume of breathing gases remains in the lungs and airways. This residual volume results in the pressure being maintained at a minimum level, such as a PEEP level as shown in pressure waveform 202. A clinician may configure the PEEP settings on a ventilator (such as through user interface 128) to maintain a specific level of PEEP, based on the respiratory condition of a patient.

Following the first exhalation phase (Exhalation 1), the ventilator system begins the inhalation phase (Inhalation 2) of the next breathing cycle. The second breathing cycle may be substantially similar or the same as the first breathing cycle, with the ventilator delivering breathing gases and allowing for exhalation as described above.

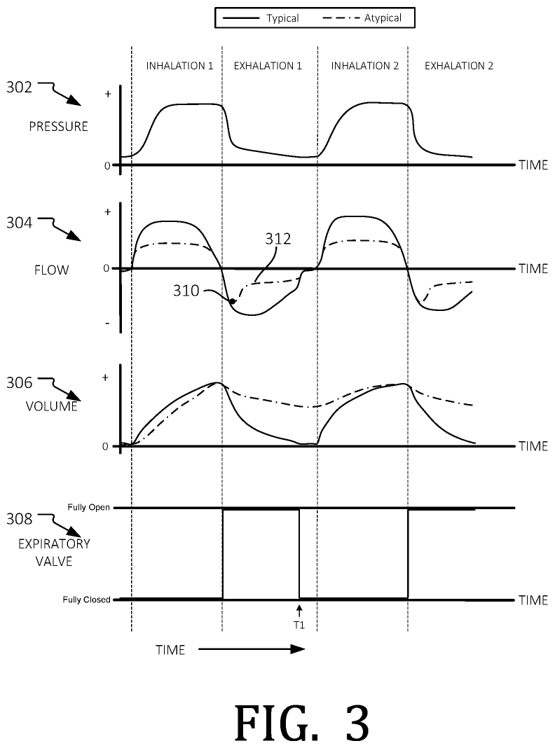

FIG. 3 illustrates the same set of waveforms depicted in FIG. 2, with the addition of example signals indicative of a patient who may be experiencing airway obstruction or collapse, such as a patient with COPD or other condition as described above. For instance, FIG. 3 includes a pressure waveform 302, a flow waveform 304, a volume waveform 306, and a valve-state waveform 308. The waveforms include a typical-patient signal (solid line) and a signal for an atypical patient (dashed line), such as a COPD patient or patient with a condition that leads to the airway collapse or obstruction discussed herein.

In examples of airway obstruction or collapse, the flow waveform 304 and volume waveform 306 may provide an indication of the obstruction or collapse. Beginning with the first exhalation phase (Exhalation 1), the expiratory valve transitions from fully closed to fully open, as indicated in the valve-state waveform 308. Similar to the behavior described in FIG. 2, the pressure in the patient's airway may drop precipitously, as the expiratory valve opens and airflow out of the lungs rapidly increases (becomes more negative). Accordingly, the signal in the pressure waveform 302 for the typical patient and the atypical patient may be substantially the same.

As described above, in examples where the atypical patient may have COPD or a similar respiratory condition, the loss of elasticity in respiratory tissue or increased airway resistance, combined with the steep pressure gradient between the lungs and expiratory valve, may result in airway obstruction or collapse. As indicated by the atypical patient signal depicted in the flow waveform 304 and the volume waveform 306, when the airways collapse, the airflow out of the lungs may markedly decrease as a result of the now obstructed airway. For instance, during exhalation, the exhalation flow rate may have a peak or maximum 310 followed by a lower substantially plateaued section 312. (Of note, while the flow rate is negative, the maximum flow rate is marked as the maximum absolute value of the exhalation flow rate.) As a result, for the atypical patients, the maximum exhalation flow rate 310 during an exhalation phase may be substantially higher than the average exhalation flow rate for the exhalation phase. The difference between the maximum exhaled flow rate 310 and the average exhalation flow rate may be referred to as the maximum-to-average exhaled flow rate difference and may be used as a breathing metric to identify a collapse or obstruction condition. For instance, a maximum-to-average exhaled flow rate difference greater than a threshold may be indicative of a collapse or obstruction condition that may be improved by increasing the airflow resistance during exhalation.

Due to the lower flow rate, the volume of breathing gases expelled from the lungs is also substantially reduced due to the limited flow out of the lungs. For instance, the volume signal in the volume waveform 306 may not return to a baseline value or beginning value (e.g., 0 in the volume waveform 306) corresponding to when the gases were first delivered to the lungs. As an example, the total exhaled volume of breathing gases may be less than the maximum delivered or inhaled volume of breathing gases. Such a difference between the exhaled volume and the maximum inhaled volume of breathing gases may be referred to as the exhaled-to-inhaled volume difference and may be used as a breathing metric to identify a collapse or obstruction condition. For instance, an exhaled-to-inhaled volume difference greater than a threshold may be indicative of a collapse or obstruction condition that may be improved by increasing the airflow resistance during exhalation.

In examples, changes in pressure may not be discernible during airway collapse as measured at the ventilator. Thus, the typical-patient signal and the atypical-patient signal in the pressure waveform 302 may be similar to examples where a patient does not experience airway obstruction or collapse. In other examples, pressure sensor measurement data may present discernible changes in the pressure waveform, and these pressure changes may serve as detectable indications of airway obstruction or collapse.

As the first breathing cycle concludes, the ventilator may still fully close the expiratory valve at time T1 in order to maintain PEEP. At the onset of the subsequent breathing cycle and inhalation phase (Inhalation 2), the ventilator may again begin to supply oxygen or other breathing gases to the patient. As the pressure rises, the airway obstruction or collapse may be released, which allows airflow into the lungs. Depending on the configuration of the ventilator or the particular ventilator mode, however, the airflow may be limited due to the residual volume of breathing gases remaining in the lungs from the previous incomplete exhalation. As a result, in some examples, the patient may not receive the intended oxygen during each breathing cycle, the lungs may not be adequately ventilated (which may affect gas exchange within the lungs), the lungs may begin to overfill with breathing gases or other health risks or complications may result.

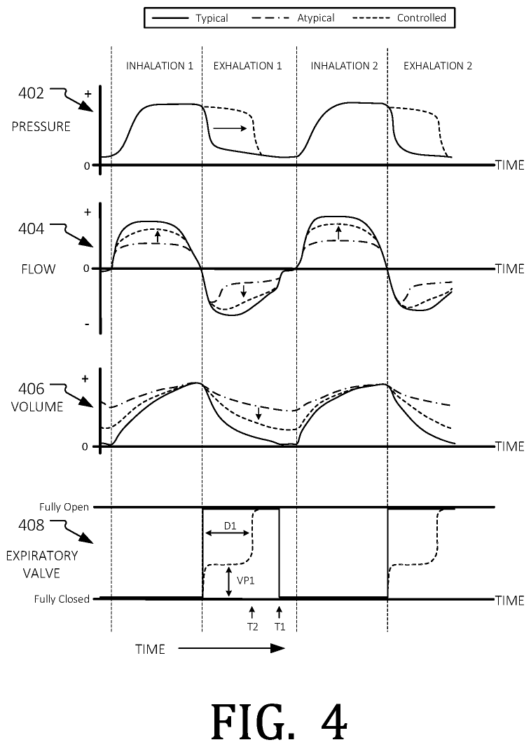

FIG. 4 depicts another set of waveforms similar to FIGS. 2-3. For instance, FIG. 4 includes a pressure waveform 402, a flow waveform 404, a volume waveform 406, and a valve-state waveform 408. In addition to the typical-patient signal and the atypical patient signal, the waveforms of FIG. 4 depict a controlled-patient signal for an atypical patient that is ventilated using the expiratory valve adjustments of the present technology, which increase the airflow resistance during the expiratory phase as compared to typical ventilation. For instance, the present technology may help reduce or eliminate the occurrence of airway obstruction or collapse and maintain sufficient airflow into and out of the lungs by controlling the closure profile of the expiratory valve during exhalation.

As described herein, sensors within the ventilator may allow the ventilator to detect airway obstruction or collapse. For example, the ventilator may measure and record physiologic breathing parameters (such as flow, volume, and/or pressure) over one or more breathing cycles and detect a trend that indicates airway obstruction or collapse. The ventilator may then respond to the obstruction or collapse, such as by controlling the closure of the expiratory valve to adjust airflow resistance. In FIG. 4, the controlled-patient signals indicate the effect on physiologic breathing parameters that may result from changes to the expiratory valve airflow resistance using the present technology to create the pursed-lip effect.

The ventilator may detect unanticipated reductions in airflow that occur during exhalation, based on trends in measurement data acquired during a set of exhalation phases (such as Exhalation 1, Exhalation 2, and subsequent exhalations). For example, the ventilator may acquire sensor measurement data over multiple breathing cycles and derive an average breathing waveform or breathing metrics that may be analyzed for trends in one or more physiologic breathing parameters, such as gas flow and volume. Additionally or alternatively, the ventilator may detect a trend indicating insufficient volume of breathing gases delivered to the patient during inhalation or insufficient volume of breathing gases exhaled from the patient during exhalation. Additional discussion regarding the detection of indications of the airway collapse or obstruction of the type of patients discussed herein (e.g., COPD patients).

In response to these indications, the ventilator may adjust the closure profile of the expiratory valve as illustrated in the valve-state waveform 408. For example, at the onset of exhalation, the ventilator may partially open the expiratory valve to first valve position VP1 to achieve a particular airflow resistance R1 (depicted in valve-state waveform 408). The ventilator may hold the expiratory valve at this level of resistance for a duration D1 to allow the patient to more fully complete an exhalation. In some examples, at time T2 the ventilator may fully open the expiratory valve as the exhalation may be fully complete at that time. In other examples, the ventilator may maintain the airflow resistance at R1 with the expiratory valve in position VP1 until time T1, when the expiratory valve may be fully closed to maintain PEEP.

The different valve positions (VP) and durations (D) for which the expiratory valve is controlled may be referred to as the closure profile of the expiratory valve. Different closure profiles may be used to cause different airflow resistances at different times of the exhalation phase. While the closure profile depicted in the valve-state waveform 408 shows only two steps (e.g., one step to valve position VP1 and another step to fully open), other closure profiles may include additional steps or may include a continuous closure rate or profile for the expiratory valve. In some examples, the closure profile of expiratory valve causes the expiratory valve to be at least 25%, 50%, 75%, or 90% closed for an initial duration of the exhalation phase. For instance, the expiratory valve may be at or above those closure percentages for at least the first 25%, 40%, 50%, 60%, or 70% of the exhalation phase. As additional airflow resistance is desired, the closure profile can be adjusted or set to increase one or both of the closure percentage or the closure duration. In other examples, the closure or opening rate may be adjusted as part of the adjustment to the closure profile.

Arrows are depicted in the pressure waveform 402, the flow waveform 404, and the volume waveform 406 to indicate the effect that expiratory valve closure may have on the physiologic breathing parameters. In the pressure waveform 402, the pressure initially begins to drop as the expiratory valve begins to open. However, due to the increased resistance created by the expiratory valve remaining only partially open (rather than fully opening), pressure may be substantially maintained in the exhalation limb (as indicated by the arrow) and the lungs, such that the airways in the lungs may be held open. This effect may be similar to the way airway pressure is maintained by purse-lipped breathing. Accordingly, the pressure remains at a higher level for a longer duration of the exhalation phase.

As depicted in the pressure waveform 402 and the flow waveform 404, with the airway held open, gas flow may be increased, and a greater volume of breathing gases may be expelled during exhalation (as indicated by the arrows). For instance, the volume signal for the controlled-patient signal in the volume waveform 406 returns closer to the baseline than that of the atypical patient signal. As such, a larger volume of gas is exhaled from the patient during the exhalation phase. As discussed above, such a result is counterintuitive because for typical patients, an increased resistance to gas flow would decrease the flow and the total volume of gas exhaled from the patient. But, for the types of atypical patients discussed herein, the additional resistance holds airways open, which allows for additional gas to escape the lungs during exhalation. In some examples and ventilator modes, the gas flow and volume of breathing gases delivered to the patient during inhalation may also be increased due to the lungs being more fully depleted during exhalation.

To achieve the increased gas flow during exhalation, the ventilator may use sensor measurement data collected over the span of one or more additional breathing cycles to further adjust the expiratory valve airflow resistance. For example, based on analyzed sensor data collected from a set of additional breathing cycles (e.g., 5-10 breaths), the ventilator may increase or decrease the airflow resistance from R1 to second resistance value R2 to further improve exhalation by changing the closure profile of the expiratory valve to a closure with second valve position VP2 that is more closed that the first profile position VP1.

In some examples, the expiratory valve airflow resistance may be adjusted more linearly, or in a more constant manner, rather than in a single discrete step or set of coarse steps as depicted in valve-state waveform 408. For example, the expiratory valve airflow resistance and valve position may be gradually changed from the fully closed to fully open state over the course of a defined interval during exhalation. In examples, the rate of change (or slope) of the change in position may be determined by a clinician, pre-configured as part of the ventilator configuration, or determined automatically by the ventilator in response to sensor measurement data or other information.

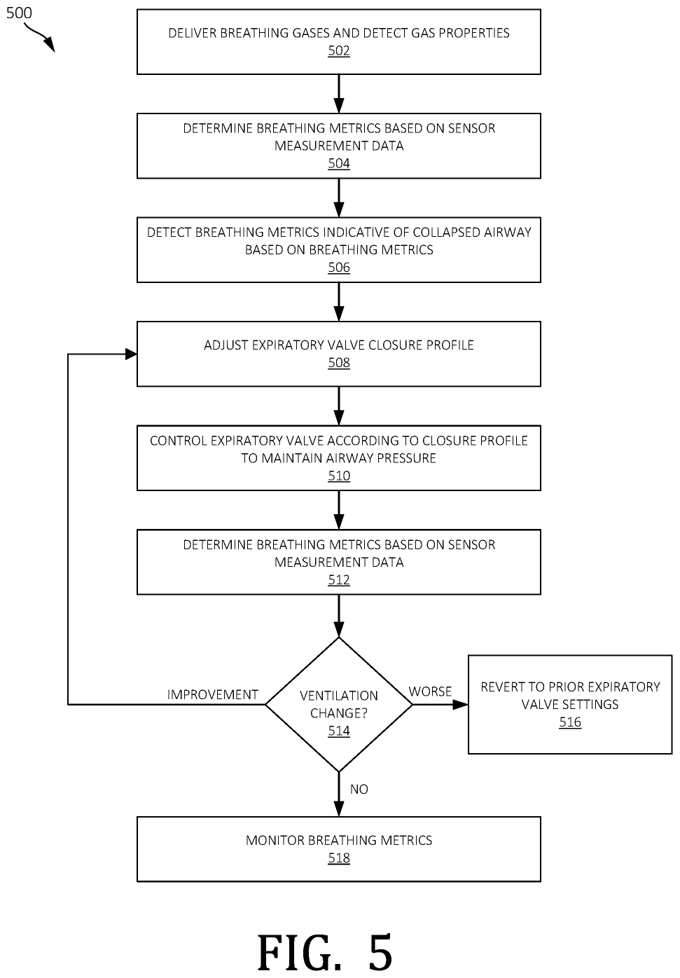

FIG. 5 is a flow diagram illustrating a method 500 for controlling a ventilator expiratory valve to improve ventilation for a patient susceptible to an obstructed or collapsed airway condition. The method 500 may be performed by a ventilator (such as ventilator 112) or by a system associated with the ventilator, such as external medical devices or similar devices or systems. The method 500 may be performed by a processor within the ventilator (such as by processor 124) or associated system, or may be performed by other suitable elements, modules, systems, or combinations thereof. The method may rely on the use of memory or storage elements (such as memory 126) during processing or analysis or for data retention or storage.

Prior to method 500 beginning, the ventilator may be initialized. The initialization may include powering on the ventilator and connecting the patient (such as patient 102) to the ventilator by way of a tubing system (such as tubing system 107). In examples, the initialization may include a calibration of systems or subsystems within the ventilator (such as sensors 122, or other elements within the ventilator). In other examples, the initialization may include receiving configuration information or preferences from a clinician (such as by user interface 128). Examples of configuration information or preferences may include ventilation mode (e.g., mandatory, intermittent, spontaneous), expiratory valve settings, PEEP settings, respiratory rate, breathing gas volume, breathing gas humidity and temperature, alert conditions, display settings, or other settings which may or may not directly relate to patient respiration. In examples, ventilator initialization may include establishing communication with any external medical devices or other systems or devices associated with the ventilator.

At operation 502, the ventilator provides ventilation to the patient by delivering breathing gases to the patient for one or more breaths. The gases are delivered according to the ventilation mode and/or settings of ventilator. As the ventilator provides ventilation, gas properties (e.g., physiological breathing parameters) of the breathing gases may be measured, detected, or otherwise derived or determined through the use of sensors. For example, the gas properties may include gas pressure and gas flow rate. As discussed above, the gas flow rate may be used to determine or calculate the volume of gas that is delivered and/or exhaled from the patient over a particular breath phase.

Sensors within the ventilator or associated system may collect measurement data of gas properties such as flow, pressure, temperature, humidity, or other types of data over time. The ventilator may use flow sensor data, along with timing information, to further derive the volume of breathing gases delivered to the patient during inhalation and the volume of breathing gases exhaled from the patient during exhalation. The ventilator may store the sensor measurement data, data derived from the sensor measurement data (e.g., volume), and/or associated timing data for further processing. For example, the ventilator may store measurement data collected from the sensors over one or more breathing cycles as waveforms, such as the waveforms discussed above.

At operation 504, the ventilator or associated system may determine breathing metrics based on gas properties detected in operation 502. The breathing metrics may be based on the gas properties detected over multiple breaths. In an example, an average breathing waveform for the gas properties is generated from a plurality of breaths, and the average waveform may be updated as new data is measured or derived. The ventilator may apply signal filtering to sensor measurement data or average waveform data. For instance, low-pass, high-pass, band-pass, notch, other type of filter, or a combination thereof, may be applied to the sensor measurement data or average flow waveforms prior to further processing, analysis, comparison, or other type of operation. Additionally or alternatively, other types of signal processing that improve signal-to-noise ratio (SNR), or other signal or noise characteristics, may be applied to the sensor measurement data or average flow waveforms. Examples may include non-linear filtering techniques.

In some examples, an average breathing waveform may be derived by first segmenting the sensor measurement waveforms into phases of the breathing cycle. For instance, an algorithm executed on a processor may determine the beginning of inhalation and exhalation phases of a sensor measurement waveform and may segment the sensor measurement waveform into corresponding groups of inhalation segments and exhalation segments. In some examples, the sensor measurement waveforms may be segmented into pairs of inhalation and exhalation phases for breaths, where each segment contains all the measurement data from one inhalation phase and one adjacent exhalation phase (e.g., the exhalation phase occurring immediately after the corresponding inhalation phase). The inhalation segments, exhalation segments, or paired inhalation/exhalation segments may then be averaged within each segmented group. For example, the segmented exhalation phases may be time-aligned and an average may be calculated at each corresponding time point across the individual segments. The result of this operation is an average exhalation waveform. Similar operations may be carried out on the inhalation segments and paired inhalation/exhalation segments. In examples, this type of operation may be considered an ensemble average. In other examples, other segmenting and averaging techniques may be applied to determine average breathing waveforms. The ventilator or associated system may store the average breathing waveforms, sensor measurement data, or other associated data in memory for later processing or analysis.

In some examples, the ventilator may collect, store, or display portions of a waveform for any of the physiologic breathing parameters. For example, the ventilator may only store measurements from one or more flow sensors associated with the exhalation limb but may not store measurements from flow sensors associated with the inhalation limb. In other examples, the ventilator may measure and store flow sensor data from both inhalation and exhalation limbs, but may only display one or the other, or may only use one or the other in further processing. For example, the ventilator or associated system may determine average breathing waveforms for only the exhalation portion of the breathing cycle, based on measured data collected during the exhalation phase only (e.g., the average exhalation waveform described above). In still other examples, the ventilator may only analyze measurement data from a subset of the flow sensors (e.g., flow sensors associated with the exhalation limb) to determine the degree of expiratory valve closure needed to achieve adequate airflow resistance.

Additionally or alternatively, the ventilator may determine numeric breathing metrics associated with the physiologic breathing parameters for display and/or analysis. For example, the ventilator may determine and store numeric values for the volume of gases delivered during inhalation and the volume of gases vented during exhalation and may display these values on the ventilator display (such as on display 130). In some examples, the breathing metrics may include metrics output from frequency analysis, phase analysis, timing analysis, statistical analysis or other type of processing or analysis carried out within the ventilator or associated system. Breathing metrics based on the physiologic breathing parameters may also be determined. For instance, breathing metric such as the maximum-to-average exhaled flow rate difference and/or exhaled-to-inhaled volume difference may be generated. These breathing metrics, related data, or other associated data may be stored in memory for later processing or analysis.

At operation 506, based on the breathing metrics determined in operation 504, the ventilator or associated system detects an indication of airway obstruction or collapse. The ventilator or associated system may use a combination of flow, volume, pressure, or waveform measurement data from another physiologic breathing parameter to detect airway collapse. For example, the ventilator or associated system may detect abrupt or subtle changes in exhalation airflow to detect airway obstruction or collapse. In some examples, the ventilator or associated system may detect changes in both airflow and exhalation volume or may detect insufficient airflow or exhalation volume as indications of airway obstruction or collapse. In other examples, the ventilator or associated system may rely on pressure waveform measurements alone or in combination with other waveforms or metrics to detect an indication of airway obstruction or collapse. Upon detection of indications of airway obstruction or collapse, the ventilator may provide a visual alert to the ventilator display, may sound an audible alert, or may provide both visual and audible alerts.

The breathing metrics of maximum-to-average exhaled flow rate difference and/or exhaled-to-inhaled volume may be used in the determination of the collapsed airway condition. For instance, a maximum-to-average exhaled flow rate difference greater than a threshold may be indicative of a collapse or obstruction condition that may be improved by increasing the airflow resistance during exhalation. An exhaled-to-inhaled volume difference greater than a threshold may be indicative of a collapse or obstruction condition that may be improved by increasing the airflow resistance during exhalation. If one or both of these breathing metrics exceed their respective thresholds, a collapse condition may be identified.

Other analyses on the measured waveform data to detect an indication of airway obstruction or collapse may also be performed. For example, the ventilator or associated system may utilize thresholding techniques as part of the detection process, where detection may be based on a threshold in amplitude, timing, power, energy, frequency, other metric, or a combination of these signal characteristics. In other examples, the ventilator or associated system may use aspects of waveform morphologies as part of the detection process, or may use pattern recognition, pattern classification or other techniques to detect an indication of airway obstruction or collapse. In some examples, statistical measures may be used alone or in combination with the above-described analyses to detect an indication of airway collapse or obstruction.

At operation 508, the ventilator or associated system determines, adjusts, or sets the closure profile of the expiratory valve to mitigate the suspected airway obstruction or collapse. The expiratory valve closure profile may be correlated with a specific airflow resistance to be used during exhalation to help maintain airway patency. The expiratory valve closure profile may include parameters or settings for the degree to which the expiratory valve is open/closed (such as valve position VP1 in FIG. 4) and the length of time the expiratory valve should be held in the partially open/closed state (such as duration D1 in FIG. 4), among other possible settings or parameters. In examples where the expiratory valve is gradually opened over the course of exhalation (rather than in a single discrete step or set of coarse steps), the expiratory valve closure profile may include parameters associated with this type of valve actuation, such as the effective slope of the variation.

The ventilator or associated system may determine expiratory valve closure profile parameters based on preconfigured settings or values provided to the ventilator or associated system. For example, the ventilator or associated system may implement a configured or default step change in expiratory valve airflow resistance. In other examples, the expiratory valve closure profile may be based on statistical data gathered from a population of patients. For example, the ventilator or associated system may compare flow, pressure, volume, or other sensor data (or derived data) to stored statistical data for physiologic breathing parameters of the population of patients. The expiratory valve settings may then be determined based on the comparison. As a further example, the ventilator or associated system may use a look-up table to determine expiratory valve closure profile parameters based on comparisons to statistical data or based on characteristics of the sensor data or breathing metrics. In other examples, the expiratory valve closure profile may be based on heuristic or other empirical data available to the ventilator or associated system. In examples where the expiratory valve airflow resistance has been previously adjusted to mitigate an airway obstruction or collapse in a ventilated patient, the ventilator or associated system may consider previous changes to expiratory valve settings, or may implement fixed, preconfigured changes to expiratory valve settings.

At operation 510, the ventilator controls the exhalation valve according to the closure profile, which was set or adjusted in operation 510, to affect expiratory valve airflow resistance and help maintain appropriate pressure within the exhalation limb and patient airways. The ventilator may control the expiratory valve by electrical, mechanical, pneumatic, or other types and combinations of control mechanisms. In examples, a processor within the ventilator or associated system may determine expiratory valve closure profile parameters as described and may further translate the expiratory valve settings to control signals needed to adjust closure of the expiratory valve. In other examples, a processor transmits control signals to an expiratory valve control module (such as expiratory controller 116), which in turn adjusts closure of the expiratory valve according to the closure profile.

At operation 512, the ventilator or associated system may again determine breathing metrics based on sensor measurement data. Operation 512 may be substantially the same as operation 504 with the exception that the breathing metrics are generated for breaths occurring after the adjustments to the expiratory valve operation (e.g., controlling the expiratory valve according to the adjusted closure profile). As such, the breathing metrics generated at operation 512 may be referred to as updated breathing metrics. The updated breathing metrics, which may include average waveforms, generated at operation 512 allow for analysis of the effect of the expiratory valve modulation performed in operation. The updated breathing metrics, such as average breathing waveforms, sensor measurement data, and/or other data, may be stored in memory to facilitate later processing or analysis.

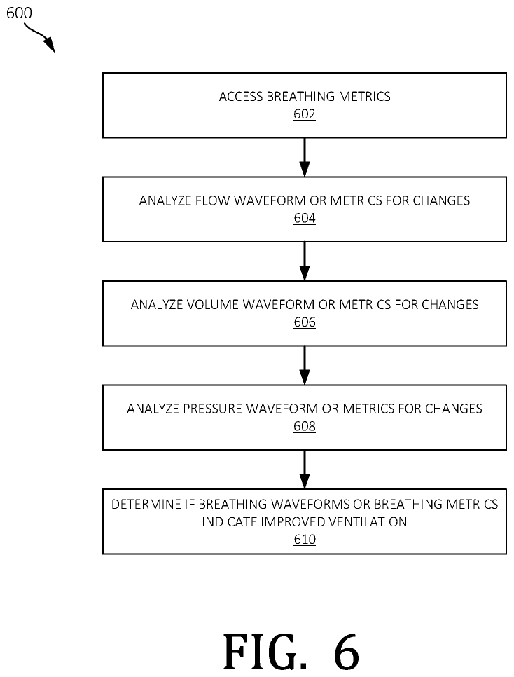

At operation 514, the ventilator or associated system determines whether the adjustment to the closure profile of the expiratory valve airflow resistance resulted in improved ventilation, such as in improved airflow or increased volume of exhaled breathing gases. In examples, the determination may be based on: (1) whether airflow increased or decreased following a change in expiratory valve airflow resistance; (2) whether volume of exhaled breathing gases increased or decreased following a change in expiratory valve airflow resistance; and/or (3) whether the pressure remained above a pressure threshold for a threshold duration of time. Additional details of the process of determining whether the airflow or exhaled volume has improved are outlined in FIG. 6 and described below.

In examples where a change in airflow resistance due to the adjusted closure profile worsens ventilation, the ventilator may revert to a previous expiratory valve setting or settings at operation 516 or change to a different closure profile that has a lower airflow resistance. In examples where the adjustment to the closure profile did improve the airflow or exhaled volume, the ventilator or associated system may use the updated sensor measurement data to determine whether additional changes in airflow resistance may further improve airflow or exhaled volume. For instance, the ventilator or associated system may return to operation 508 to further adjust or set an updated closure profile for the expiratory valve based on updated average breathing waveforms or breathing metrics (generated at operation 512).

Operations 508 through 514 may be implemented as a loop, in which an algorithm executed on the ventilator attempts to achieve continuously improved ventilation of the patient. For example, an algorithm may search for a level of expiratory valve airflow resistance that maximizes airflow or volume of breathing gases exchanged in the lungs of the patient. The algorithm may adjust the degree of expiratory valve closure in fine steps, coarse steps, variable steps, or fixed, pre-defined steps, according to the configuration of the ventilator. In examples, the algorithm may consider the effect of previous changes to expiratory valve airflow resistance, such as the degree of improvement to any of the physiologic breathing parameters (e.g., flow, volume, pressure) due to previous change in expiratory valve airflow resistance, degree of the previous changes to expiratory valve airflow resistance, whether the previous change to expiratory valve airflow resistance improved or worsened ventilation of the patient, or other considerations. Such an algorithm may rely on average breathing waveforms, rather than direct sensor measurement data, since average breathing waveforms may reduce instability in the optimization loop. However, in some examples, the algorithm may incorporate sensor measurement data or other breathing metrics rather than, or in addition to, average breathings waveforms.

In some examples, at operation 514, the ventilation may be determined to have stayed substantially the same (e.g., gas flow or exhaled volume may be within a threshold range from the prior gas flow and exhaled volume metrics). For instance, no further improvements are evident by the adjustment to the closure profile of the expiratory valve airflow resistance. In such instances, the method 500 flows to operation 518, where the ventilator or associated system may make no further adjustments to the closure profile, but new sensor measurement data may be collected and analyzed on an ongoing basis. For example, updated breathing waveforms and breathing metrics based on the new sensor measurement data may be generated. The ventilator or associated system may continue to monitor the updated average breathing waveforms and breathing metrics for indications of airway obstruction or collapse. If future indications of airway collapse occur, method 500 may be repeated.

FIG. 6 is a flow diagram illustrating a method 600 for evaluating changes in airflow, exhaled volume, or other physiologic breathing parameters following modulation of expiratory valve airflow resistance. The method 600 may apply to, or be performed as part of, operation 514 of method 500 depicted in FIG. 5. The method 600 may be performed by a ventilator (such as ventilator 112) or by a system associated with the ventilator, such as external medical devices or similar devices or systems. In examples, the method may be performed by a processor within the ventilator (such as processor 124) or associated system, or may be performed by other suitable elements, modules, systems, or combinations thereof. The method may rely on the use of memory or storage elements (such as memory 126) during processing or analysis or for data storage.

At operation 602, the ventilator or associated system accesses breathing metrics, such as average waveforms, that were calculated and stored during other operations. For example, operation 504 describes an example of how breathing metrics may be calculated and stored for processing or analysis.

At operation 604, a flow waveform or flow metrics may be processed or analyzed to determine changes in gas flow resulting from modulation of expiratory valve airflow resistance. The determination may be based on signal processing or analysis techniques. In examples, the processing or analysis may require comparisons of updated breathing metrics collected after modulation of airflow resistance (such as those stored at operation 512) with breathing metrics collected before modulation of airflow resistance (such as those stored at operation 504).

In some examples, prior to analyzing average flow waveforms, signal filtering may be applied to the average flow waveforms, in addition to any signal filtering which may have been applied to the waveform data in previous operations or at the circuit level. For example, any type of low-pass, high-pass, band-pass, notch, other type of filter, or any combination thereof, may be applied to the average flow waveforms prior to further processing, analysis, comparison, or other type of operation. Additionally or alternatively, other types of signal processing that improve signal-to-noise ratio (SNR), or other signal or noise characteristics, may be applied to the average flow waveforms. Examples may include non-linear filtering techniques.

To determine whether gas flow has improved following modulation of airflow resistance, the flow waveforms or metrics may be analyzed for changes in peak amplitude, average amplitude, areas under the curves, signal energy, signal power, spectral content, or other signal metric suitable for comparing flow metrics collected before and after airflow resistance modulation. In examples, the processing or analysis may include statistical measures such as mean error, mean absolute error, mean squared error (MSE), root mean square error (RMSE), or other error statistic appropriate for determining differences between flow metrics collected before and after modulation of expiratory valve airflow resistance.

At operations 606 and 608, substantially similar processing or analysis may be performed on the pressure waveform or metrics and the volume waveform or metric as was performed at operation 604 on average flow waveforms. Additionally or alternatively, other physiologic breathing parameters may be processed or analyzed, such as measurement data associated with pulse oximetry, capnometry, or other metrics. In examples, processing or analysis of average breathing waveforms (such as any of those described above) may be performed on inhalation waveform segments, exhalation waveform segments, paired inhalation/exhalation waveform segments, or other segmentations or groupings of sensor measurement waveforms or data.

At operation 610, processing or analysis may be performed to determine whether changes in the breathing waveforms or associated metrics analyzed in operations 604-608 have improved overall patient ventilation. For instance, if the exhalation flow increases after the change in the expiratory valve, such an increase may be an indication of improved patient ventilation. Similarly, if the volume of exhaled gas increases after the change in the expiratory valve, such an increase may be an indication of improved patient ventilation.

In examples, thresholding techniques may be utilized to determine whether changes in the breathing metrics are significant. For instance, a gas flow change of 2 L/min during exhalation may be set as a threshold for determining significant changes in gas flow because of changes in expiratory valve airflow resistance. In a first scenario, processing and analysis may determine that an improvement of 1 L/min of gas flow has occurred between average flow waveforms collected before and after the airflow resistance change. In this scenario, the change would not be regarded as significant or an improvement, since the 1 L/min change does not exceed the threshold of 2 L/min. In a second scenario, processing and analysis may determine that an improvement of 4 L/min of airflow has occurred between average flow waveforms collected before and after the airflow resistance change. In this scenario, the change is significant and may be determined to be an improvement in patient ventilation.

In other examples, a variety of thresholds may be used to determine whether significant changes have occurred in any of the average breathing waveforms as a result changes in expiratory valve airflow resistance. In still other examples, any combination of average breathing waveforms or breathing metrics may be considered in the determination. For example, a change in expiratory valve airflow resistance may not be considered significant, unless changes in both exhalation flow and exhalation volume meet corresponding thresholds.

In some examples, where average waveform sample size permits, statistical measures of significance may be applied in determining whether changes in expiratory valve airflow resistance have improved patient ventilation. For example, ANOVA, MANOVA, or other tests of statistical significance may be performed on one or more of the physiologic breathing parameters, associated average breathing waveforms, breathing metrics, or any other data processed or analyzed as described above.

In examples, results of the processing or analysis may indicate that changes in the expiratory valve airflow resistance have caused regression in one or more of the physiologic breathing parameters. For instance, if expiratory valve airflow resistance is increased too much, the resulting pressure may hold the patient's airway sufficiently open, but the respiratory effort required to overcome the increased resistance (to maintain airflow) may exceed what the patient can provide. Consequently, analysis of the average flow waveforms may indicate a decrease in airflow after the increase in airflow resistance. Such an outcome may be significant, and may require further action, such as a reduction in expiratory valve airflow resistance. Such action may be performed automatically, such as by a processor or other system within the ventilator, or manually, such as by a clinician.

Those skilled in the art will recognize that the methods and systems of the present disclosure may be implemented in many manners and as such are not to be limited by the foregoing aspects and examples. In other words, functional elements being performed by a single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for controlling an expiratory valve of a medical ventilator to improve ventilation, the method comprising:
delivering, by the ventilator, breathing gases for multiple breaths;
detecting a flow rate of the breathing gases during a first inhalation phase;
based on the detected flow rate during the first inhalation phase, calculating an inhaled volume of breathing gases delivered during the first inhalation phase;
detecting a flow rate of the breathing gases during a first exhalation phase;
based on the detected flow rate during the first exhalation phase, calculating an exhaled volume of breathing gases delivered during the first exhalation phase;
based on the exhaled volume being less than the inhaled volume by at least a threshold value, determining an indication of a collapsed airway; and
based on determining the indication of the collapsed airway, adjusting a closure profile of the expiratory valve for a second exhalation phase wherein the closure profile of the expiratory valve increases airflow resistance for the second exhalation phase.

2. The method of claim 1, wherein determining the indication of the collapsed airway is further based on a maximum-to-average exhaled flow rate difference.

3. The method of claim 1, wherein the second closure profile of the expiratory valve causes the expiratory valve to be at least 50% closed for at least a first 25% of the second exhalation phase.

4. The method of claim 1, wherein the expiratory valve is external to the ventilator.

5. The method of claim 4, wherein the expiratory valve is positioned between a wye and a patient interface.

6. The method of claim 1, wherein the closure profile of the expiratory valve causes the expiratory valve to be at least 25% closed for at least a first 50% of the second exhalation phase.

7. The method of claim 1, further comprising:
detecting a flow rate of breathing gases for the second exhalation phase;
based on the detected flow rate of breathing gases for the second exhalation phase, determining that the closure profile of the expiratory valve reduced an exhaled volume for the second exhalation phase; and
based on the closure profile of the expiratory valve reducing the exhaled volume, reverting the closure profile of the expiratory valve to a prior closure profile.

8. A medical ventilation system comprising:
a ventilator including an inspiratory port and an expiratory port;
an expiratory valve positioned external to the ventilator;
a flow sensor;
a processor; and
memory storing instructions that when executed by the processor cause the medical ventilation system to perform operations comprising:
delivering breathing gases for a first breath;
controlling the expiratory valve according to a first closure profile during a first exhalation phase of the first breath;
detecting, by the flow sensor, an exhaled gas flow rate during the first exhalation phase;
determining that the exhaled gas flow rate during the first exhalation phase is indicative of a collapsed airway during the first exhalation phase;
based on determining that the exhaled gas flow rate is indicative of the collapsed airway, setting a second closure profile of the expiratory valve for a second exhalation phase wherein the second closure profile of the expiratory valve increases airflow resistance for the second exhalation phase of a second breath; and
controlling the expiratory valve according to the second closure profile during the second exhalation phase.

9. The medical ventilation system of claim 8, wherein the operations further comprise:
determining a maximum exhaled gas flow rate;
determining an average exhaled gas flow rate; and
wherein determining that the exhaled gas flow rate during the first exhalation phase is indicative of the collapsed airway is based on a difference between the maximum exhaled gas flow rate and the average exhaled gas flow rate.

10. The medical ventilation system of claim 8, wherein the operations further comprise:
determining a delivered gas volume during an inhalation phase prior to the first exhalation phase;
determining an exhaled gas volume during the first exhalation phase; and
wherein determining that the exhaled gas flow rate during the first exhalation phase is indicative of the collapsed airway is based on a difference between the delivered gas volume and the exhaled gas volume.

11. The medical ventilation system of claim 8, wherein the second closure profile of the expiratory valve causes the expiratory valve to be at least 25% closed for at least a first 50% of the second exhalation phase.

12. The medical ventilation system of claim 8, wherein the second closure profile of the expiratory valve causes the expiratory valve to be at least 50% closed for at least a first 25% of the second exhalation phase.

13. The medical ventilation system of claim 8, wherein the operations further comprise:
detecting, by the flow sensor, an exhaled gas flow rate during the second exhalation phase;
based on the exhaled gas flow rate during the second exhalation phase being higher than the exhaled gas flow rate for the first exhalation phase, setting a third closure profile of the expiratory valve for a third exhalation phase wherein the third closure profile of the expiratory valve increases the airflow resistance for the second exhalation phase compared to the first exhalation phase; and
controlling the expiratory valve according to the third closure profile during the third exhalation phase.

14. The medical ventilation system of claim 8, wherein the operations further comprise:
detecting, by the flow sensor, an exhaled gas flow rate during the second exhalation phase;
based on the exhaled gas flow rate during the second exhalation phase being lower than the exhaled gas flow rate for the first exhalation phase, setting a third closure profile of the expiratory valve for a third exhalation phase wherein the third closure profile of the expiratory valve decreases the airflow resistance for the second exhalation phase compared to the first exhalation phase; and
controlling the expiratory valve according to the third closure profile during the third exhalation phase.

15. The medical ventilation system of claim 8, wherein the operations further comprise:
detecting, by the flow sensor, an exhaled gas flow rate during the second exhalation phase; and
based on the exhaled gas flow rate during the second exhalation phase being within a threshold range of the exhaled gas flow rate for the first exhalation phase, controlling the expiratory valve according to the second closure profile during a third exhalation phase.
